(19) United States
(12) Patent Application Publication
(10) Pub. No.: US 2025/0114462 A1
(43) Pub. Date: Apr. 10, 2025

(54) HYDROPHILIC FUNCTIONALIZED PARTICLES IN THE DELIVERY OF HYDROPHOBIC DRUGS

(71) Applicant: NEW JERSEY INSTITUTE OF TECHNOLOGY, Newark, NJ (US)

(72) Inventors: Somenath Mitra, Bridgewater, NJ (US); Mohammad Saiful Islam, Newark, NJ (US)

(73) Assignee: NEW JERSEY INSTITUTE OF TECHNOLOGY, Newark, NJ (US)

(21) Appl. No.: 18/911,710

(22) Filed: Oct. 10, 2024

Related U.S. Application Data
(60) Provisional application No. 63/543,328, filed on Oct. 10, 2023.

(51) Int. Cl. A61K 47/59; A61K 9/51; A61K 31/343; A61K 31/42; A61K 31/437; A61K 31/567; A61K 31/573

(52) U.S. Cl. CPC A61K 47/593; A61K 9/51; A61K 31/343; A61K 31/42; A61K 31/437; A61K 31/567; A61K 31/573

(57) ABSTRACT

Described herein are hydrophilic functionalized polymers (HFPs), methods of preparing such HFPs. Also described are methods of solubilizing hydrophobic drugs using such HFPs. HFP-drug composites, and methods of administering such HFP-drug composites.

### CROSS-REFERENCE TO RELATED APPLICATIONS

This application claims priority to U.S. Provisional Application Ser. No. 63/543,328, filed Oct. 10, 2023, the entirety of which is hereby incorporated by reference herein.

### FIELD OF THE DISCLOSURE

The present disclosure relates to drug formulation. In particular, the present disclosure relates to the use of hydrophilic functionalized particles in drug formulation to enhance drug solubility.

### BACKGROUND

Many pharmaceutical compounds are hydrophobic and/or relatively insoluble in water. The therapeutic effects of such compounds are significantly reduced due to low aqueous, membrane, chemical and enzymatic solubility. These poorly water-soluble drugs are unable to fully release in the body such as in the gastrointestinal tract, thus, contributing to their low and variable bioavailability. These compounds with low solubility are classified as BCS class II and class IV drugs. It is reported that over 70% of discovered drugs and active entities are categorized in these two classes—poorly soluble, with reduced dissolution rates that suffer from formulation challenges. Therefore, it is important to explore new methods to optimize dosage forms for improved aqueous dissolution which may increase drug absorption. The inability to hydrogen bond in an aqueous medium is among the major reasons behind low aqueous solubility. Increasing solubility not only improves drug absorption but can also reduce the dosage needed to achieve the same therapeutic effect, thus reducing side effects and toxicity.

Typically, dissolution for insoluble and BCS class II and class IV drugs are improved by techniques such as micronization, spray-dried dispersion, chemical modification via complexation, salt formation, and the use of cryogenic techniques such as freeze-drying. Nano or semi micro-sizing of drug excipients also have positive effects for dissolution. These are carried out by methods such as spray drying, wet and jet milling, high pressure homogenization, anti-solvent precipitation, and supercritical fluid technology. The control of morphology is an important factor in these applications. Precipitation processes are effective methods for synthesizing micron and submicron hydrophobic drug particles. Nanoparticles have been used in drug delivery and controlled release applications where they have been attached to drug molecules via covalent or non-covalent bonding.

There is an unmet need to improve the solubility of various drugs for drug formulation.

### SUMMARY

Various embodiments of the present disclosure relate to enhancing the dissolution of insoluble and/or hydrophobic drugs by incorporating hydrophilic nanoparticles in the drug crystal structure. While not wishing to be bound by any particular theory, it is believed that such hydrophilic nanoparticles attract water to improve dissolution. In various embodiments, enhancing the solubility of such insoluble and/or hydrophobic drugs improves the dissolution rate, kinetic solubility and/or efficacy of such drugs.

Polymeric nanomaterials are of great interest in diverse sectors due to their unique properties, however many polymers cannot be used in drugs due to their toxicity and safety concern. PLA (polylactic acid) and PLGA (polylactic co glycolic acid) are FDA-approved materials that have received much attention in biomedical research application due to their biocompatibility and biodegradability. They are used widely in pharmaceutical and many other fields. This acid-based material is a highly active enantiomer found mostly in crystal form. The natural polymer PLA and PLGA are synthesized from sugarcane, rice, corn, wheat as well as fruits and milk. Recently, both of these renewable polymers have been used in the pharmaceutical sector due to their strong hydrophobicity and acidic properties.

PLA and PLGA, with their small pore size/volume and high porosity surface, make them attractive carriers of drugs, proteins, and genes. Different processing methods allow the production for PLA films, laminates, particles, and fibers, which have been synthesized via solution casting, extrusion, and spray drying. PLA and PLGA are hydrophobic materials. Because PLA and PLGA are FDA approved, they have been used in drug delivery system due to their smart linkages with drug particles, which has relied on physical and chemical degradation that controls rate of release. Meanwhile, the physical properties of PLA and PLGA also depend on their initial molecular weight, D/L ratio, lactide to glycolide ratio, particle size, water exposure (as both a reactant and a plasticizer), and storage temperature.

In accordance with various embodiments of the present disclosure, highly hydrophilic functionalized particles are disclosed. In one or more embodiments, the hydrophilic functionalized particles could be incorporated into hydrophobic drugs to enhance dissolution. In one or more embodiments, the hydrophilic functional particles could be PLA and/or PLGA. Other exemplary polymers include polycaprolactone (PCL), polyglycolic acid (PGA), polypropylene oxide (PPO), polyester, chitosan, chitin, polyanhydrides (PA) and/or poly (2-hydroxyethyl methacrylate) (PHMA). The hydrophilic functionalized particles (HFP) could immobilize nano and micro hydrophobic drug crystals. The HFP-drug composites or cocrystals shows higher aqueous solubility and dissolution behavior as well as improved lipophilic character for the bioavailability development.

In one or more embodiments, a method to produce hydrophilized particles and incorporating into water insoluble hydrophobic drugs to enhance solubility is disclosed. The method could include the step of reacting a PLA and/or PLGA powder formulation with an oxidizing agent to form a mixture. The method could also include the steps of homogenizing the mixture and subjecting the substantial homogenized mixture to a functionalization and then incorporating them into a drug particle.

In one or more embodiments, a process to produce HFP from original PLA and PLGA is disclosed. The process could employ a microwave-based technique where an oxidation step could be used to produce HFP from original PLA and PLGA. The microwave induced hydrophilization technique could include the step of reacting the nano PLA with strong oxidizers. In this manner, the oxygen content can increase over 50% along with hydrophilicity as measured by solubility in polar solvents such as acetone, ethanol and dimethyl sulfoxide.

An increase in the hydrophilicity of PLA and PLGA based HFP can synthesize HFP-drug composites from hydrophobic drugs that could have high aqueous solubility. The oxidation process enhances the oxygen content of the PLA/PLGA and hydrophilicity for different drug delivery applications especially the delivery of hydrophobic drugs.

In general, formulated HFP has several advantages for oral drug delivery. Hydrophilic formulations of PLA/PLGA have generally been made using additives such as stabilizers or surfactants that are not very effective and can make them cytotoxic. In one or more embodiments, hydrophilic formulations are made without any additives. An advantage is that the physical and chemical stability of the formulated drug improved for storage and polymer incorporated into solid dosage forms (drugs) with a relatively straightforward process. The hydrophilic HFP matrices where the drug release is controlled either by matrix swelling or by the matrix slow dissolution and polymer-drug conjugates where drug release is chemically controlled.

In one or more embodiments, the microwave functionalization approach could involve controlling the pressure, temperature, time, and the concentration of the material used has a large impact on its hydrophilization. The prepared material is accurately monitored for functionalization. In one or more embodiments, the polymer is functionalized in the presence of an oxidizing agent such as an acid. In one or more embodiments, the material used is purified with nitric acid and then functionalized with 3:1 ratio of sulfuric acid and nitric acid. It will be understood that other ratios and acids could be employed. The process also controlled the acid concentration effect for functionalization. A strong oxidizing agent could predominantly functionalize the surface of the polymer.

Drug-HFP composites using hydrophilized PLA and PLGA can be made by several approaches. In one or more embodiments, anti-solvent crystallization that uses non-solvent water is typically different from polymer solvent or drug solvent uses for drug recrystallization. In one or more embodiments, functional PLA and PLGA polymers are produced that carry higher oxygen concentration compared to their pure form. For example, the functionalization processes described herein can increase the surface oxygen content of PLA or PLGA to greater than 30%, as measured by scanning electron microscopy/energy-dispersive X-ray spectrometry (SEM-EDS) analysis.

Furthermore, relatively small amounts of HFP can provide large increases in aqueous solubility of hydrophobic drugs. For example, the drug-HFP composite can comprise less than 10 wt % HFP or less than 5 wt % HFP, such as 0.5 to 2 wt % HFP.

Other aspects of the present disclosure relate to methods of administering such drug-HFP composites to a patient in need thereof. In various embodiments, the composite is delivered orally, intranasally, transdermally or by injection.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed hydrophilic functional particles and associated systems and methods, reference is made to the accompanying figures, wherein:

FIG. 1 shows images for elemental study of the surface of neat/original PLA (left) and the microwave synthesized oxygenated functional nfPLA (right). The right image highlights the powder form of nfPLA generated with an increase in pore surfaces and the increased wt % of oxygen concentration. An average 50% increase in oxygen concentration (represented by O in the images) on the surface of nfPLA generated data show successful hydrophilization;

FIG. 2 illustrates images of PLGA (left) and another competitive functional copolymer nfPLGA (right). SEM-EDS data shows a significant increase in hydrophilicity due to almost 100% increased number of oxygen wt % concentration at the surface of nfPLGA. Increased pore surface is also visible in the right image in the generated PLGA through microwave acid digested PLGA;

FIG. 3 shows scanning (SEM) images for different types of drug-functional polymer/HFP composites.

FIG. 4 shows the graphical depictions illustrating spectral difference for pure PLA (FIG. 4a) and PLGA (FIG. 4b) compared to nf-PLA and nf-PLGA, respectively.

FIG. 5 illustrates a contact angle measurement study for PLA compared to nf-PLA. The observed angle found to up to zero for nfPLA referred to as production of highly hydrophilic polylactic acid polymer; and

FIGS. 6A-6E are graphical depictions of the in vitro dissolution profile for drug-nanofunctional polymer composites. The schematic defines the increased dissolution profile observed that highlights the significant enhancement in dissolution rate properties.

### DETAILED DESCRIPTION

Exemplary embodiments are directed to hydrophilic functionalized PLA and PLGA. It should be understood that embodiments could generally be applied to other hydrophilic functional particles. Other exemplary polymers for functionalization include, but are not limited to, polycaprolactone (PCL), polyglycolic acid (PGA), polypropylene oxide (PPO), polyester, chitosan, chitin, polyanhydrides (PA) and/or poly (2-hydroxyethyl methacrylate) (PHMA).

In one or more embodiments, a hydrophobic particle is subjected to functionalization (e.g. surface functionalization) to provide the hydrophilic functionalized particle.

In one or more embodiments, the functionalization includes increasing the surface oxygen content of the particle. However, other hydrophilic surface functionalization is also within the scope of the present disclosure.

In various embodiments, the hydrophilic functionalized particle is a nanoparticle. In one or more embodiments, the hydrophilic functionalized particle is a nanoparticle but the drug form (e.g. crystal) is not nano-sized. In other embodiments, both the hydrophilic functionalized particle and the drug are nano-sized.

In one or more embodiments, either PLA or PLGA could be formulated into a hydrophilic form. The PLA or PLGA is reacted with a strong oxidizer. The functionalized PLA or PLGA could be combined with a drug, such as a hydrophobic drug, to create a functionalized PLA-drug composite or a functionalized PLGA-drug composite as well as cocrystallized polymer drug composites too.

In various embodiments, the drug is a substantially hydrophobic drug. In one or more embodiments, the drug is a BCS class II drug (e.g. high permeability, low solubility). In one or more embodiments, the drug is a BCS class IV drug (e.g. low permeability, low solubility). Exemplary drugs include, but are not limited to, griseofulvin, sulfamethoxazole, megestrol acetate, apixaban, dexamethasone and combinations thereof.

In one or more embodiments, a process to generate the functionalized PLA-drug composite or the functionalized PLGA-composite is disclosed. The process could include the step of mixing a powder form of either PLA or PLGA with one or more oxidizing agents. In one or more embodiments, the oxidizing agent comprises an acid such as sulfuric acid and/or nitric acid. Other oxidizing agents include, but are not limited to, hydrogen peroxide (H₂O₂) and ozone (O₃). In one or more embodiments, the oxidizing agent is an oxygen-containing oxidizing agent and/or the functionalization is performed in the present of an oxygen-containing co-reagent (e.g. water). Other oxidizing agents can also be used that can provide different hydrophilic functionalizations. The mixed formulation could be sonicated for homogenous distribution of the powder particles in the solution. The ultrasonication process could create nanoparticles dispersions. After ultrasonication, the mixed formulation could undergo microwave functionalization, which could be operated at suitable parameters. In one or more embodiments, the microwave functionalization is at a temperature of 50-90° C., such as about 50° C., about 60° C., about 65° C. about 70° C., about 75° C., about 80° C., about 90° C. or any range therebetween. In one or more embodiments, the microwave functionalization is at a pressure of 10-1,000 psi, such as about 10, about 20, about 30, about 40, about 50, about 60, about 70, about 80, about 90, about 100, about 150, about 200, about 250, about 300, about 350, about 400, about 500, about 600, about 700, about 800, about 900, about 1,000 psi or any range therebetween. In one or more embodiments, the microwave functionalization is at a power of 100-5,000 watts, such as about 100, about 150, about 200, about 250, about 300, about 350, about 400, about 450, about 500, about 550, about 600, about 700, about 800, about 900, about 1,000, about 1,500, about 2,000, about 3,000, about 4,000, about 5,000 watts or any range therebetween. In one or more embodiments, suitable parameters include 60° C. temperature, 200 psi pressure, and 400 watt power in a Teflon sealed container.

Disclosed is a formulation process to make hydrophilic a polymer nanomaterial in drug delivery formulation research. In one or more embodiments, a high-power microwave instrument is employed that could pass its radiative frequency of microwave radiation into the acid dispersed sample and could excite the particle in its increased ionized form. The microwave instrument could help to generate surface oxygen that overall increases the content of oxygen concentration into the polymeric material. A process could formulate a powder form of nfPLA or a crystal/powder form of nfPLGA maintaining the original structure intact. Additionally, the particles could be controlled with a high-power probe sonication process. Both PLA and PLGA maintained a very small nanosize that was determined in Zetasizer instrumentation and maintained a stabilized form in a water colloidal solution.

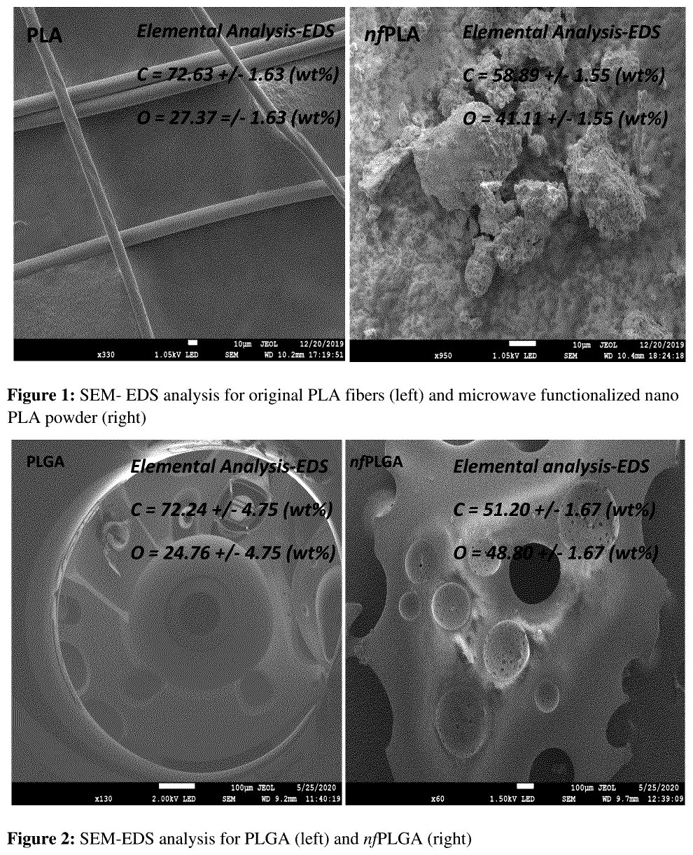

The SEM images in FIGS. 1 and 2 show the porous form into the surface of PLA and PLGA nanomaterial. In one or more embodiments, PLA and/or PLGA was incorporated with very hydrophobic BCS-II and BCS-IV drugs. The increased oxygen may create a diffusion into the single pore and may multiply that portion observed in nanofunctional PLGA.

When the drug is incorporated with HFP, the results verify higher drug loadings and the composite structure to increase in solubility. A presence of a very small quantity, such as 1-2% of hydrophilic functionalized polymer (HFP), dispersed onto the drug surfaces resulted in a dramatic improvement in solubility and hence increase dissolution rate. Accordingly, in one or more embodiments, the HFP-drug composite comprises less than 10 wt % HFP, such as 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 wt % HFP or any range therebetween.

While not wishing to be bound by any particular theory, it is possible that the increase in solubility is accomplished by generating a hydrophilic channel onto the drug crystal surfaces, causing water contact and hydrolytic degradation. It is evident from the FIG. 2c images that the crystal structure of the single drug GF and DXM remains intact within the physical integration and attachment of the nfPLGA on the surface of the cocrystals. Additionally, the nfPLGA particles may provide hydrophilic linkages over the drug surfaces which may produce a water channel among the cocrystals may led to high dispersibility and aqueous solubility. Overall, the analyses suggests that the co-formulated drugs are capable of retaining their structural and morphological integrity upon nfPLGA incorporation during antisolvent process.

### EXAMPLES & EXPERIMENTS

The materials and the methods of the present disclosure used in one or more embodiments will be described below. While the embodiments discuss the use of specific compounds and materials, it is understood that the present disclosure could employ other suitable compounds or materials. Similar quantities or measurements may be substituted without altering the method embodied below.

Acetone (AR≥99.5) obtained from Sigma-Aldrich, sulfuric acid (99.0% purity), nitric acid (99% purity), and hydrochloric acid were purchased from Fisher Scientific. Milli-Q water was obtained from the York facilities at New Jersey Institute of Technology (NJIT). PLA fibers were obtained from Carbion Inc. and PLGA crystals were obtained from Polysciences Inc. The average diameter of the supplied PLA particle was 5.0-10.0 μm and of the PLGA crystals was ˜ 5.0 μm.

Both PLA polymer particle and PLGA crystalline particle are highly insoluble particles. A microwave technique used in this embodiment modified its solubility properties significantly. The PLA fiber grinded to produce powder formulation. About 100 mg of powder form PLA then mixed with 1M of 3:1 ratio called 45 mL sulfuric acid and 15 mL nitric acid reagents. The mixed formulation then bath sonicated for 5 minutes for homogeneous distribution of powder particles in solution. The mixed sample then ran for microwave functionalization, which was operated at 70° C. temperature, 200 psi pressure, and 400-watt power in a Teflon sealed container. The sample ran in the CEM microwave instrument for 50 minutes for continuous functionalization to occur.

A high-power probe sonicator was used for the nanosization of this functional PLA and PLGA material. Temperature and power were controlled for a specified size investigation. The sample dispersed in water and sonicated for a specified time by controlling power produced very fine nanocrystal particles. Finally produced nano polymer particle filtered with membrane filter paper and dried.

A prepared nanocrystal and nano powder suspension were characterized with Malvern Zetasizer for particle size measurement. A small portion of the nano powder was dispersed in water and bath sonicated for homogeneous dispersion. The dispersed sample was analyzed in Zetasizer instrument.

An anti-solvent technique was applied for drug-polymer composites preparation. Water was used as an anti-solvent agent. 100.0 mg of griseofulvin drug sample dissolved with an acetone reagent. Functional PLA that is highly oxygen increased concentrated dissolved with acetone with mild heating (40.0° C.) added in drug solution dropwise in bath sonicator provided sufficient time for mixing. Generally, a 10.0 minutes bath sonicator was used for mixing of drug and polymer solution. A clear and cloudy solution of mixed composites was obtained and maintained for room temperature to reach normal temperature. Finally, water was added dropwise into the clear cloudy solution for precipitation to occur. The produced crystal resulted due to anti-solvent process and highly hydrophilic drug composites compared to the pure drug sample. Finally, the precipitated sample was filtered and dried in vacuum for 48.0 hours for constant weight.

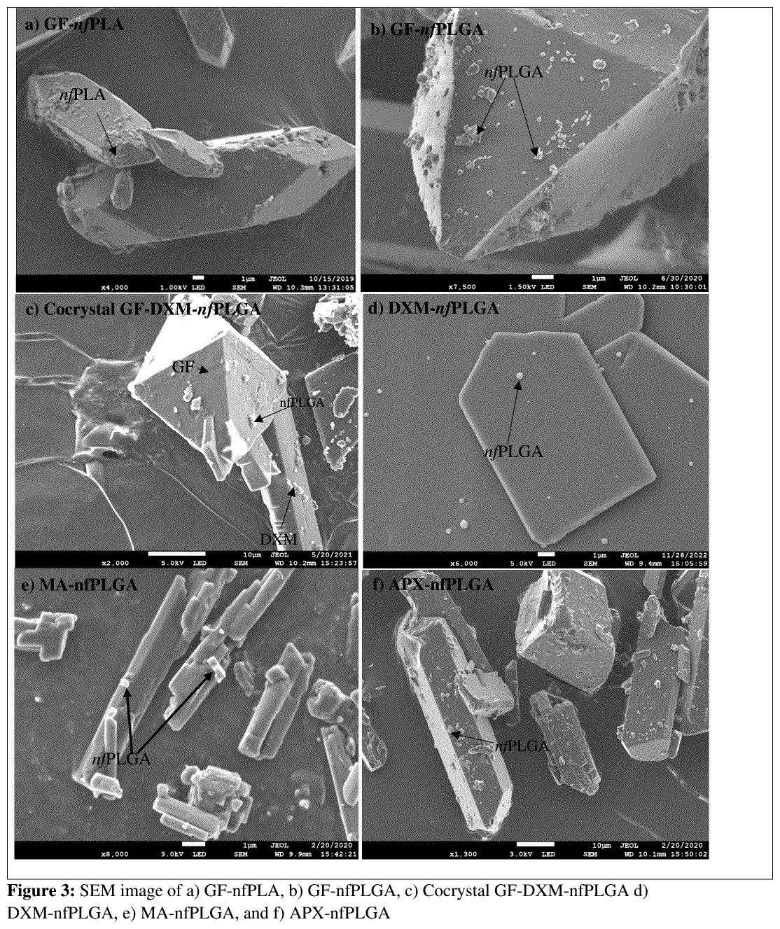

Prepared drug-polymer composites morphological characteristics with surface and structure properties was determined by a scanning electron microscope (JEOL JSM 7900F, Japan). The sample tiny quantity was dispersed in a carbon tapped aluminum pan. The samples were then coated through carbon coating for conductivity resistance using EMS Quorum sputtering coater. The corresponding images were captured at suitable magnification. FIGS. 3a-3f show the molecularly dispersed state of drug into the polymer matrices or vice versa. For griseofulvin (GF), nfPLA (FIG. 3a) and nfPLGA FIG. (3b) fine nano particles are observed to be loosely dispersed onto the drug's inner and outer surfaces to produce a hydrophilic channel for a hydrophobic drug. FIG. 3c shows the cocrystal formulation from the single drug molecules of griseofulvin (GF) and dexamethasone (DXM) with the nfPLGA incorporation. Next FIGS. 3d-f show nfPLGA particles incorporated with DXM, megestrol acetate (MA) and apixaban (APX), respectively.

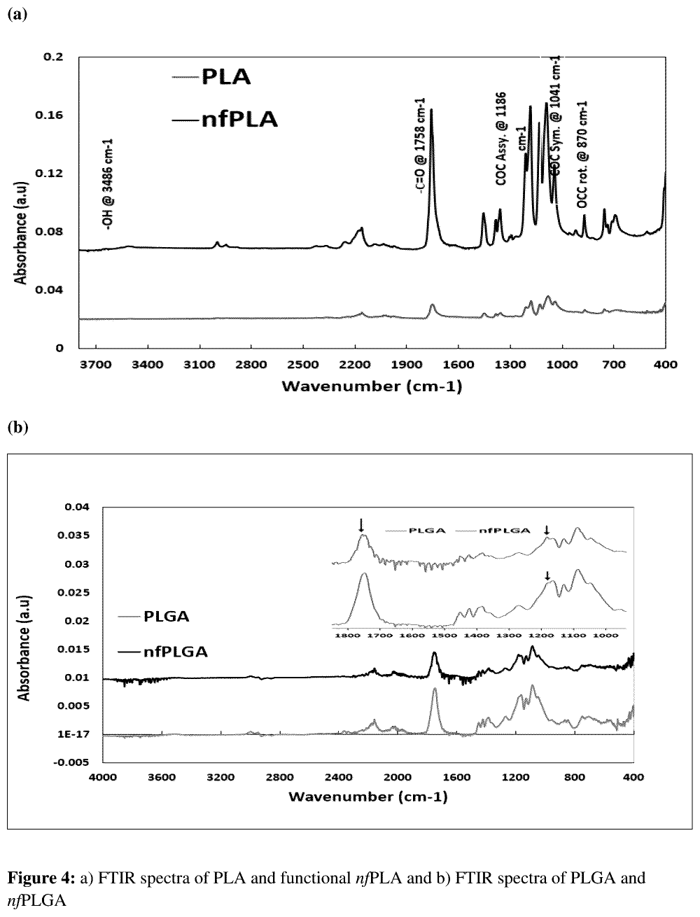

Prepared PLA and PLGA that referred to highly oxygenated carried particles and hydrophilic water insoluble functionalized polymer was characterized through FTIR analysis for hydrophilic functional development and structural peak analysis. In FIG. 4a, the top spectra are for nfPLA and the bottom spectra are for the original PLA. Similarly, in FIG. 4b, the top spectra are for nfPLGA and the bottom spectra are for the original PLGA. The FTIR analysis of FIG. 4a shows the functional nfPLA increase in intensity for carboxylated carbonyl —C═O peak significantly that demonstrates increase in oxygen functionality. For PLA, the oxygenated peak is observed at 1758 cm-1 region. Additional increase in broader peak observed at 3486 cm-1 for —OH for lactic acid monomer production or carboxylated —OH. Similarly, for nf-PLGA increased carbonyl peak intensity observed at 1737 cm-1 region, as shown in FIG. 4b. Additional increased peak intensity for nf-PLGA also observed at 1157 cm-1 referred to as ═C—O stretch.

Thermogravimetric analysis (TGA) was used to investigate the degradation of modified PLA, PLGA, and drug-polymer composites materials during heating. TGA was carried out using a Perkin-Elmer Pyris TGA system at 30.0 to 700.0° C. under a 20.0 mL/min air flow with heating rate of 10.0° C./min under air to monitor the incorporation concentration of nanofunctionalized polymer over the drug molecules respectively.

Raman spectroscopy was carried out using a Bruker Scientific DXR Raman Microscope with 532 nm wavelength laser and filter. Powder X-Ray diffraction was performed by using PANalytical EMPYREAN XRD with Cu-Kα radiation source under scanning conditions of 5.0-70.0 degrees angular range for crystallization structure monitoring.

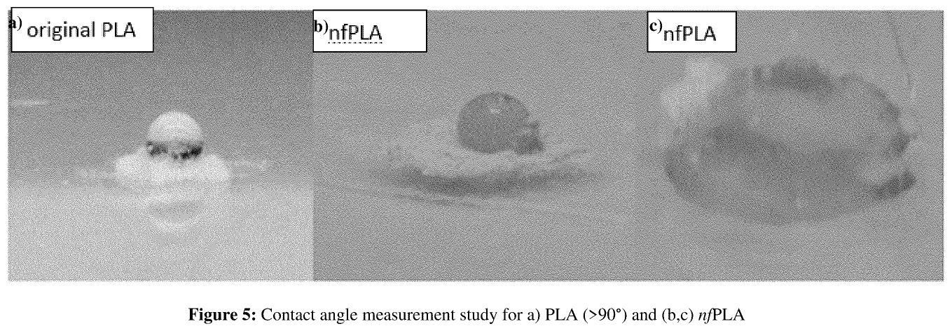

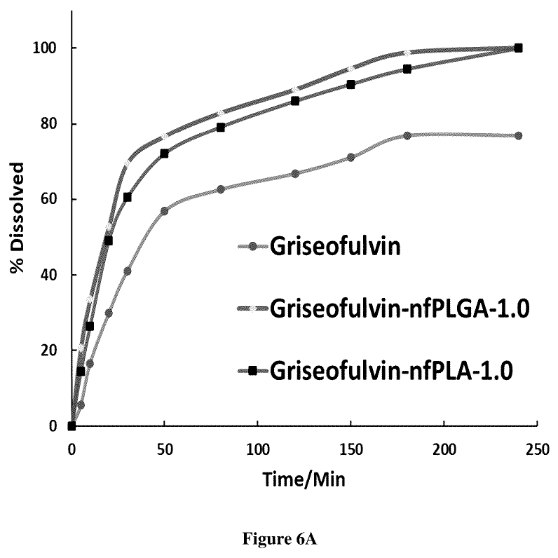

FIG. 5 shows the contact angle measurement for original PLA (FIG. 5a) compared to nfPLA (FIGS. 5b and 5c). As can be seen, the contact angle for original PLA is >90° whereas the contact angle for nfPLA approached zero.

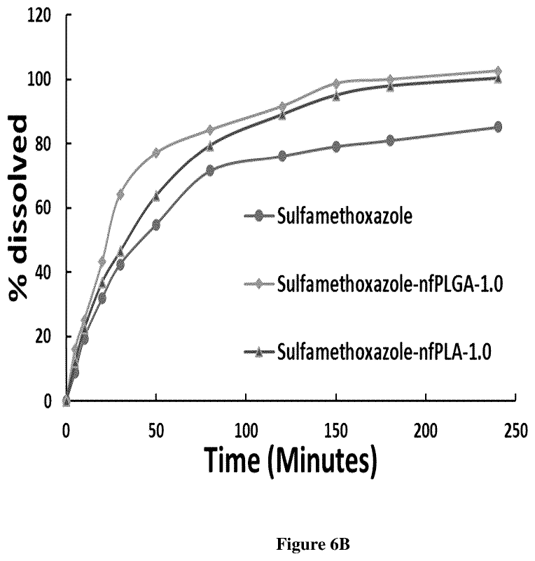

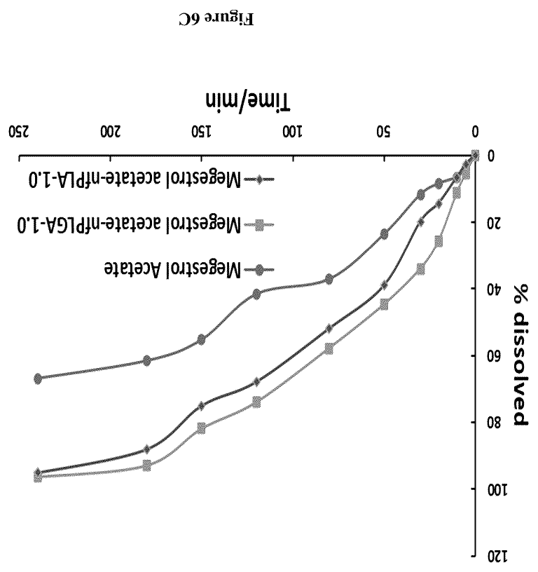

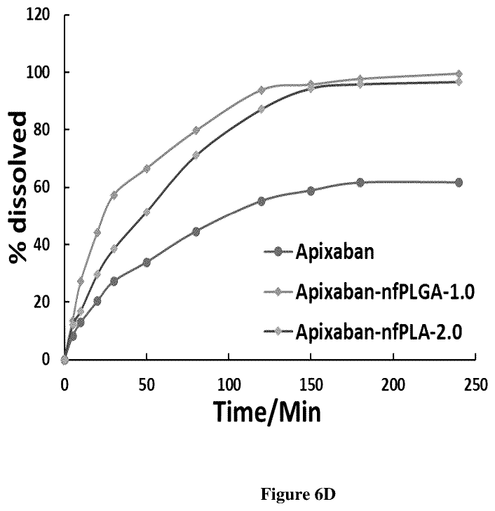

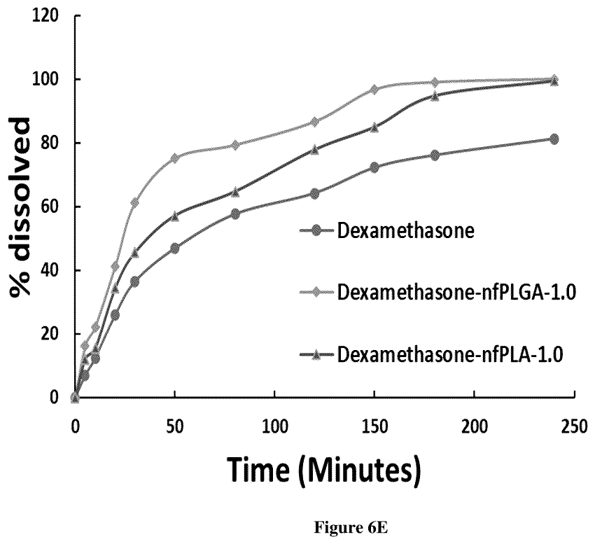

FIGS. 6A-6E and Table 1 show the dissolution data for various drugs with and without HFPs as described herein. Dissolution measurements were carried out using USP-42 paddle method with Symphony 7100 Distek dissolution instrument. Agilent 8453 model UV-Visible spectrophotometer was used at a wavelength at 295.0 nm for the reference drug Griseofulvin standard absorbance measurements that calibrated to measure the formulation polymer incorporated drugs concentration and dissolution percentage estimation.

Table 1 represents enhancement dissolution time profiles for griseofulvin nfPLA, griseofulvin-nfPLGA, cocrystals, and other types of drugs such as sulfamethoxazole (SMZ), megestrol acetate (MA), apixaban (APX) and dexamethasone (DXM) with the nfPLGA composites material respectively. The table highlight the 50% and 80% dissolution time that shows enhanced reduced time for all the nfPLA and nfPLGA incorporated drugs and cocrystal formulations. Additionally enhanced initial dissolution rate (μg/min) for all the functionalized polymer HFP incorporated drug were also presented.

TABLE 1
In vitro dissolution profile for different
drug-HFP/FNP composite formulations

| | 50% | 50% | Initial | |
|---|---|---|---|---|
| | dissolution | dissolution | dissolution | Melting |
| Drugs and | time | time | rate | Point |
| composites | (T₅₀) | (T₈₀) | (μg/min) | (° C.) |
| Griseofulvin (GF) | 40 | undissolved | 110.3 | 222.1 |
| GF-nfPLA | 20 | 82 | 210.5 | 222.5 |
| GF-nfPLGA | 18 | 63 | 266.8 | 222.6 |
| Cocrystal | 16 | 57 | 450.2 | 212.5-242.2 |
| GF-DXM-nfPLGA | | | | |
| Sulfamethoxazole | 41 | undissolved | 186.5 | 172.3 |
| (SMZ) | | | | |
| SMZ-nfPLA | 33 | 81 | 367.4 | 168.6 |
| SMZ-nfPLGA | 22 | 58 | 488.6 | 167.4 |
| Megestrol Acetate | 138 | undissolved | 84.1 | 219.8 |
| (MA) | | | | |
| MA-nfPLA | 76 | 161 | 212.6 | 218.6 |
| MA-nfPLGA | 61 | 142 | 245.7 | 217.5 |
| Apixaban (APX) | 137 | undissolved | 74.3 | 240.5 |
| APX-nfPLA | 38 | 100 | 368.2 | 238.4 |
| APX-nfPLGA | 23 | 81 | 540.1 | 236.2 |
| Dexamethasone | 66 | undissolved | 289.7 | 261.7 |
| (DXM) | | | | |
| DXM-nfPLA | 37 | 128 | 465.8 | 258.4 |
| DXM-nfPLGA | 23 | 87 | 513.0 | 257.8 |

The dissolution profile data presented in Table 1 demonstrating the 50% and 80% of dissolution of drug-polymer composites, highlights a significant reduction in dissolution time when incorporating with 0.5 to 1.0% of HFP. This result could be due to a strong interaction of polymer-drug particles that stay in solid state powder dispersed form. The polymer drug interaction can be defined due to a potential strong inter and intramolecular surface hydrogen bonding and weak van der Waals interaction into drug crystal surfaces. The weak intermolecular van der Waals interaction creates a passage for water molecules to channel into drug crystal surfaces and creates hydrogen bonding to assist in dissolving the drug composites at a faster rate compared to their pure drug molecules. The prepared drug-polymer composites and coformulated cocrystals, which are highly crystalline and retain the polymorphism properties, maintained the drugs original crystallinity and its active API form. Hence, an enhanced dissolution phenomena could essentially be observed for this type of prepared drug-nanofunctionalized polymer composite particles.

As can be seen from Table 1 and FIGS. 6A-6E, relatively low levels of HFP (e.g. nfPLA and/or nfPLGA) significantly increased the solubility of each of the tested hydrophobic drugs.

Table 2 shows the solubility for microwave functionalized PLA, and PLGA nanomaterials compared to the original PLA and PLGA materials.

TABLE 2
Solubility data for microwave and probe ultrasonicated FNP

| Functionalized nanomaterial | Acetone solubility | |
|---|---|---|
| (HFP) | (mg/mL) | Aqueous solubility |
| Original PLA/PLGA | Insoluble | Insoluble/nondisperse |
| nfPLA | 1.085 | dispersible |
| | (heat @ 40° C.) | |
| nfPLGA | 1.33 | dispersible |

Both functionalized polymer nanomaterials had increased acetone solubility compared to the original polymers, and also had increased water dispersibility.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A method of enhancing the solubility of substantially hydrophobic drugs, the method comprising:
incorporating one or more hydrophilic functionalized particles with a substantially hydrophobic drug to form a composite, wherein the one or more hydrophilic functionalized particles interact with water.

2. The method of claim 1, wherein the one or more hydrophilic functionalized particles are formed by surface functionalization of one or more hydrophobic particles.

3. The method of claim 1, wherein the one or more hydrophilic functionalized particles are formed by a process comprising:
reacting a powder formulation comprising one or more of polylactic acid (PLA) or polylactic co glycolic acid (PLGA) with an oxidizing agent to form a mixture;
homogenizing the mixture to provide a substantially homogenized mixture; and
subjecting the substantially homogenized mixture to a functionalization to produce hydrophilic functionalized particles.

4. The method of claim 3, wherein the functionalization comprises a microwave functionalization.

5. The method of claim 3, where the oxidizing agent comprises at least one acid.

6. The method of claim 1, wherein incorporating the one or more hydrophilic functionalized particles with a substantially hydrophobic drug comprises an antisolvent precipitation.

7. The method of claim 6, wherein the antisolvent precipitation provides a crystalline form of the substantially hydrophobic drug with the one or more hydrophilic functionalized particles on a surface of the drug crystal.

8. The method of claim 1, wherein the substantially hydrophobic drug is selected from a BCS class II drug and a BCS class IV drug.

9. A composite comprising one or more hydrophilic functionalized particles and a substantially hydrophobic drug.

10. The composite of claim 9, wherein the one or more hydrophilic functionalized particles comprise one or more of functionalized polylactic acid (f-PLA) or functionalized polylactic co glycolic acid (f-PLGA).

11. The composite of claim 10, wherein the one or more hydrophilic functionalized particles comprise f-PLA.

12. The composite of claim 10, wherein the one or more hydrophilic functionalized particles comprise f-PLGA.

13. The composite of claim 10, wherein the one or more hydrophilic functionalized particles comprising f-PLA or f-PLGA have a surface oxygen content of greater than 30 wt % as measured by SEM-EDS analysis.

14. The composite of claim 9, wherein the one or more hydrophilic functionalized particles comprise one or more of polycaprolactone (PCL), polyglycolic acid (PGA), polypropylene oxide (PPO), polyester, chitosan, chitin, polyanhydrides (PA) or poly (2-hydroxyethyl methacrylate) (PHMA).

15. The composite of claim 9, wherein the substantially hydrophobic drug is selected from a BCS class II drug and a BCS class IV drug.

16. The composite of claim 9, wherein the one or more hydrophilic functionalized particles comprise nanoparticles.

17. The composite of claim 9, wherein the composite comprises 0.5 to 2 wt % of the one or more hydrophilic functionalized particles.

18. The composite of claim 9, wherein the substantially hydrophobic drug is in crystalline form and the one or more hydrophilic functionalized particles are on a surface of the drug crystal.

19. A method comprising delivering the composite of claim 9 to a subject.

20. The method of claim 19, wherein the one or more hydrophilic functionalized particles comprise one or more of functionalized polylactic acid (f-PLA) or functionalized polylactic co glycolic acid (f-PLGA).

21. The method of claim 20, wherein the composite is delivered orally.

22. The method of claim 20, wherein the composite is delivered intranasally, transdermally or by injection.
